(19) United States
(12) Patent Application Publication
(10) Pub. No.: US 2025/0114378 A1
(43) Pub. Date: Apr. 10, 2025

(54) ANACARDIC ACID FOR INFLAMMATORY CONDITIONS

(71) Applicant: Vanderbilt University, Nashville, TN (US)

(72) Inventor: Subramaniam Sriram, Nashville, TN (US)

(21) Appl. No.: 18/922,313

(22) Filed: Oct. 21, 2024

Related U.S. Application Data
(63) Continuation-in-part of application No. 18/796,266, filed on Aug. 6, 2024.
Application No. 18/796,266 is a division of application No. 17/267,608, filed on Feb. 10, 2021, now Pat. No. 12,083,134.
(60) Provisional application No. 62/717,051, filed on Aug. 10, 2018.

(51) Int. Cl. A61K 31/60; A61P 29/00

(52) U.S. Cl. CPC A61K 31/60; A61P 25/28

(57) ABSTRACT

The present invention discloses a method for reducing inflammation in a subject by administering an effective amount of anacardic acid (AA) or a pharmaceutically acceptable salt thereof. Anacardic acid, represented by the chemical structure, has been found to exhibit anti-inflammatory properties when administered to a subject in need thereof. The method provides a novel approach to treating inflammation by utilizing anacardic acid or its pharmaceutically acceptable salt, offering a promising therapeutic option for managing inflammatory conditions in subjects.

### RELATED APPLICATIONS

This application is a continuation-in-part of U.S. patent application Ser. No. 18/796,266, filed Aug. 6, 2024, entitled “ANACARDIC ACID FOR NEURAL REPAIR,” which is a divisional of U.S. patent application Ser. No. 17/267,608, filed Feb. 10, 2021, entitled “ANACARDIC ACID FOR NEURAL REPAIR”, which is a 371 of International Patent Application No. PCT/US2019/046119, filed Aug. 12, 2019, entitled “ANACARDIC ACID FOR NEURAL REPAIR,” which claims the benefit of U.S. Provisional Application No. 62/717,051, filed Aug. 10, 2018, entitled “ANACARDIC ACID FOR NEURAL REPAIR.” These applications are hereby incorporated by reference in their entirety for all purposes.

### FIELD OF THE DISCLOSURE

The present disclosure relates generally to a composition comprising an anacardic acid and a process of preparing the anacardic acid.

### BACKGROUND

Psoriasis is a chronic, relapsing inflammatory skin disease influenced by environmental, genetic, and immunological factors. It affects 2% to 3% of the general population. Topical therapies continue to play a major role in managing mild to moderate psoriasis, which accounts for 80% of psoriasis cases.

Plaque psoriasis is the most common type of cutaneous psoriasis and can affect almost any region of the body, with a preference for the extensor surfaces. The pathogenesis of psoriasis is believed to result from the amplification of Th17 T lymphocytes in the skin, leading to keratinocyte proliferation, local inflammation, and subsequent skin thickening and scaling. Plaque psoriasis may typically present with large oval-circular plaques over the scalp, trunk, and extensor body surface, with an extensive amount of scaling due to the hyperproliferation of the epidermal cells. In a subset of patients, psoriasis also leads to regional arthritis, with at least 15% of patient with plaque psoriasis developing psoriatic arthritis. Most patients with plaque psoriasis may experience acute flares and relapses of plaque psoriasis, which may sometimes become sever and progress into the pustular subtype. Treatment for severe cases of psoriasis generally require oral or intravenous agents to suppress the immune system and, in some cases, the treatments target specific pathways involved in activating proteins that promote psoriasis.

Systemic administration of immunosuppressive drugs is typically reserved for patients with severe psoriasis, defined as those whose psoriatic plaques cover more than 10% of the body. Topical therapy is used for patients with mild to moderate psoriasis. Common topical agents include salicylic acid, corticosteroids, calcineurin inhibitors, and tapinarof. However, there are several issues with the currently available topical treatments.

One major concern with salicylic acid topical agents is the potential for chronic and acute systemic toxicity, with symptoms including burning of the oral mucosa, headaches, and central nervous system effects such as nausea and vomiting. These side effects limit the use of high-concentration salicylic acid over extended periods. Calcineurin inhibitors, which target T-cells and inhibit key cytokines involved in inflammation, demonstrate moderate efficacy when combined with salicylic acid preparations. Topical corticosteroids are the most commonly used agents for mild to moderate plaque psoriasis, but their effectiveness diminishes over time due to tachyphylaxis. Furthermore, corticosteroids can cause skin thinning in areas of prolonged application, limiting their use on the face, neck, and other exposed regions.

Various immune and autoimmune diseases are mediated by T lymphocytes, which have specific markers on their surface and in their cytoplasm. T cells are broadly divided into four subgroups based on the proteins they secrete or the receptors they express on their surface: (i) TH1 cells, (ii) TH2 cells, (iii) TH17 cells, and (iv) T regulatory cells. Targeting a specific subtype of T cells allows for more precise regulation of particular autoimmune diseases.

Anacardic acid has been shown to reduce the production of Th17 cytokines in lymphocytes, which can positively affect diseases regulated by Th 17-secreting lymphocytes, such as inflammatory bowel disease, psoriasis, psoriatic arthritis, rheumatoid arthritis, multiple sclerosis, and optic neuritis. Anacardic acid is a naturally occurring compound found in cashew nutshells, cashew fruit and nuts, and other fruits such as mango and apple. Chemically, anacardic acid consists of salicylic acid substituted with an alkyl chain of 15 or 17 carbon atoms, which can be saturated or unsaturated. Natural anacardic acid is a mixture of saturated and unsaturated molecules and exists in four isoforms, along with other organic compounds such as cardols and cardanols. The isolation of purified anacardic acid typically involves organic solvents, which complicates making the compound safe for human consumption.

Therefore, there is a need to develop a synthetic route for the production of anacardic acid and to create effective compositions for the treatment of inflammatory skin disorders.

### BRIEF SUMMARY

The present disclosure provides an enhancement to conventional treatments, at least in part by introducing a novel method for treating psoriasis using an anacardic acid composition. The present disclosure relates to a composition that may comprise an anacardic acid or pharmaceutical acceptable salt thereof as an active agent and a vegetable oil as a diluent. In some embodiments, the vegetable oil may include, but is not limited to, corn oil, coconut oil, peanut oil, mustard oil, sesame oil and/or combinations thereof. The present disclosure also relates to a process of preparing an anacardic acid. Further, the present disclosure relates to use of a composition in treatment of inflammatory bowel disease, rheumatoid arthritis, multiple sclerosis, optic neuritis, and inflammatory disorders of skins. Moreover, the present disclosure relates to a method of treating inflammatory bowel disease, rheumatoid arthritis, multiple sclerosis, optic neuritis, and inflammatory disorders of skins.

In some aspects, the techniques described herein relate to a method of reducing inflammation in a subject in need thereof, wherein the method includes administering to the subject an effective amount of anacardic acid (AA) having the structure:

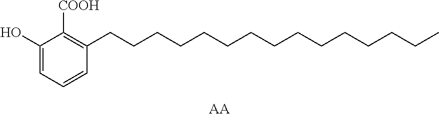 [SMILES: CCCCCCCCCCCCCCCc1cccc(O)c1C(=O)O ; "AA"]

or a pharmaceutically acceptable salt thereof.

In some aspects, the techniques described herein relate to a method, wherein AA or a pharmaceutically acceptable salt thereof is administered to the subject.

In some aspects, the techniques described herein relate to a method, wherein the subject has been diagnosed as having an inflammatory disorder.

In some aspects, the techniques described herein relate to a method, wherein the inflammatory disorder is inflammatory bowel disease, rheumatoid arthritis, multiple sclerosis, optic neuritis, inflammatory disorders of the skin, or combinations thereof.

In some aspects, the techniques described herein relate to a method, wherein the inflammatory disorder is psoriasis.

In some aspects, the techniques described herein relate to a method, wherein AA or a pharmaceutically acceptable salt thereof is administered with a pharmaceutically acceptable carrier.

In some aspects, the techniques described herein relate to a method, wherein AA is administered topically, and the pharmaceutically acceptable carrier is a vegetable oil.

In some aspects, the techniques described herein relate to a method, further including administering a composition including the effective amount of AA or a pharmaceutically acceptable salt thereof of 0.001%-1.00%.

In some aspects, the techniques described herein relate to a method, wherein the vegetable oil is a corn oil, coconut oil, peanut oil, mustard oil, sesame oil, or combinations thereof.

In some aspects, the techniques described herein relate to a method, further including administering a composition including the effective amount of AA or a pharmaceutically acceptable salt thereof of 0.5-2 mg/mL.

In some aspects, the techniques described herein relate to a method, wherein administering a composition including the effective amount of AA or a pharmaceutically acceptable salt thereof of 0.5-1.0 mg/mL.

In some aspects, the techniques described herein relate to a method, wherein administering a composition including the effective amount of AA or a pharmaceutically acceptable salt thereof of 1-2 mg/mL.

In some aspects, the techniques described herein relate to a method of reducing inflammation in a subject in need thereof, wherein the method includes administering to the subject an effective amount of anacardic acid (AA) or a pharmaceutically acceptable salt thereof.

In some aspects, the techniques described herein relate to a method, wherein the subject has been diagnosed as having an inflammatory disorder.

In some aspects, the techniques described herein relate to a method, wherein the inflammatory disorder is psoriasis.

In some aspects, the techniques described herein relate to a method, wherein the inflammatory disorder is inflammatory bowel disease, rheumatoid arthritis, multiple sclerosis, optic neuritis, inflammatory disorders of the skin, or combinations thereof.

In some aspects, the techniques described herein relate to a method, wherein AA or a pharmaceutically acceptable salt thereof is administered with a diluent.

In some aspects, the techniques described herein relate to a method, wherein AA is administered topically, and the diluent is a vegetable oil.

In some aspects, the techniques described herein relate to a method, further including administering a composition including the effective amount of AA or a pharmaceutically acceptable salt thereof of 0.001%-1.00%.

In some aspects, the techniques described herein relate to a method, further including administering a composition including the effective amount of AA or a pharmaceutically acceptable salt thereof of 0.5-2.0 mg/mL.

Numerous objects, features, and advantages of the embodiments set forth herein will be readily apparent to those skilled in the art upon reading of the following disclosure when taken in conjunction with the accompanying drawings.

### BRIEF DESCRIPTION OF THE DRAWINGS

FIG. 1 depicts a structure of anacardic acid (AA) according to an aspect of the present disclosure.

FIG. 2 depicts an exemplary Scheme 1 for a method of preparing an anacardic acid (AA) according to an aspect of the present disclosure.

FIG. 3 depicts a pathway by which anacardic acid may inhibit expression of IL-17 and provide therapeutic effects on plaque psoriasis according to an aspect of the present disclosure.

FIGS. 4A-4B depict quantification of IL-17 inhibition according to methods and compositions of the present disclosure.

### DETAILED DESCRIPTION

The terminology as set forth herein is for description of the embodiments only and should not be construed as limiting of the disclosure as a whole. As used in the description of the disclosure and the appended claims, the singular forms “a”, “an”, and “the” are inclusive of their plural forms, unless contraindicated by the context surrounding such.

The disclosure is inclusive of the compounds described herein in any of their pharmaceutically acceptable forms, including isomers (e.g., diastereomers and enantiomers), tautomers, salts, solvates, polymorphs, prodrugs, and the like. In particular, if a compound is optically active, the disclosure specifically includes each of the compound's enantiomers as well as racemic mixtures of the enantiomers. It should be understood that the term “compound” includes any or all of such forms, whether explicitly stated or not (although at times, “salts” are explicitly stated).

A subject, as defined herein, can be any animal. In one instance the subject is a vertebrate animal, and in a more particular instance the subject is a mammal, such as a domesticated farm animal (e.g., cow, horse, pig) or pet (e.g., dog, cat). In another instance, the subject is a human. The subject may also be a subject at risk of developing a myelin related disease.

As used herein, the term “treating” can relate to reversing, alleviating, inhibiting the progress of, or preventing a specific disorder or condition, prophylaxis of a specific disorder or condition, or alleviation of the symptoms associated with a specific disorder or condition and/or preventing or eliminating said symptoms.

“Pharmaceutically acceptable” as used herein means that the compound or composition is suitable for administration to a subject for the methods described herein, without unduly deleterious side effects in light of the severity of the disease and necessity of the treatment.

“Pharmaceutically acceptable salt” as used herein refers to a salt formed by the addition of a pharmaceutically acceptable base to anacardic acid. The phrase “pharmaceutically acceptable salt” connotes salts commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, y-hydroxybutyric, galactaric, and galacturonic acids. Suitable pharmaceutically acceptable base addition salts of the AA compounds described herein may include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine), and procaine may be used form base addition salts of the compounds described herein. All of these salts may be prepared by conventional means from the corresponding compounds described herein by reacting, for example, the appropriate acid or base with the compound.

The term “therapeutically effective” is intended to qualify the amount of the agent that will achieve the goal of decreasing disease severity while avoiding adverse side effects such as those typically associated with alternative therapies. An “effective amount” on the other hand, is an amount sufficient to provide a significant chemical effect.

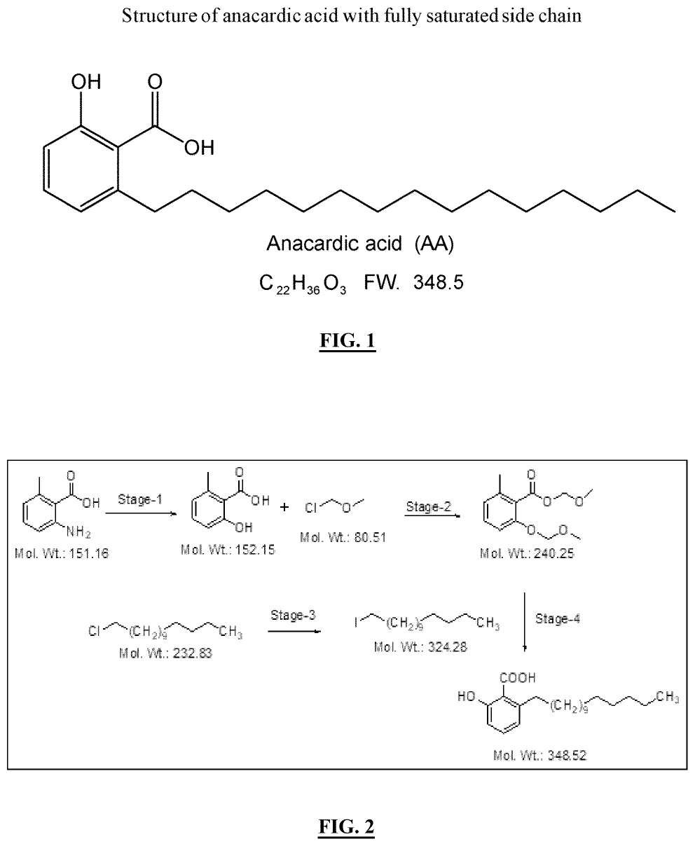

Referring now to FIGS. 1-2, various aspects may now be described of a method of reducing inflammation by administering an effective amount of anacardic acid (AA) or a pharmaceutically acceptable salt thereof.

FIG. 1 depicts a structure of anacardic acid (AA) according to an aspect of the present disclosure. AA is a non-isoprenoid long chain phenolic acid primarily obtained from the nut shells of the plant Anacardium occidentale that is native to South America. In an exemplary aspect, AA formulated to include a fully saturated side chain as depicted in structure (I) below:

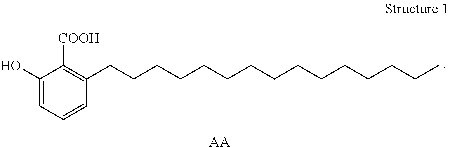 [SMILES: CCCCCCCCCCCCCCCc1cccc(O)c1C(=O)O ; "AA"]

The solvent extracted cashew nut shell liquid (CNSL) may be understood as a rich source of many long chain phenolic compounds including various anacardic acids. Three major anacardic acids may be found in CNSL. These three major forms may be the monoene, the diene, and the triene. The fully saturated form, referred to as AA, may be present in negligible amount. Taken together, the monoene, the diene, the triene, and AA may be referred to herein as the natural anacardic acids (“NAAs”).

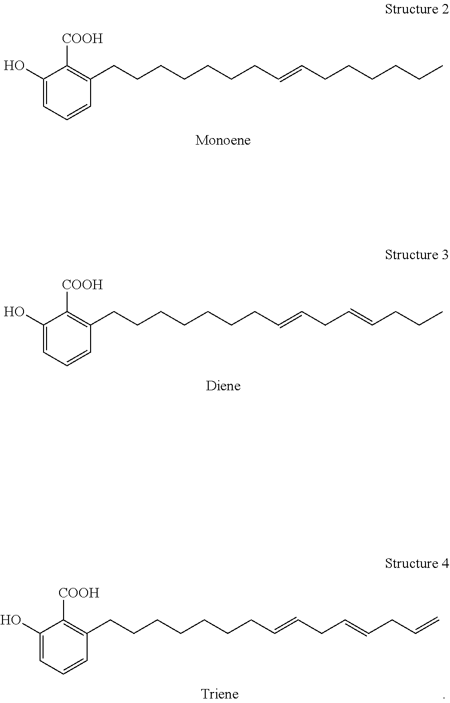 [SMILES: CCCCCC/C=C/CCCCCCCc1cccc(O)c1C(=O)O ; CCC/C=C/C/C=C/CCCCCCCc1cccc(O)c1C(=O)O ; C=CC/C=C/C/C=C/CCCCCCCc1cccc(O)c1C(=O)O]

Although the present disclosure refers to the use of AA for treating inflammation in a subject, it should be understood that any of the NAAs may be used in the methods described herein.

In some aspects, a method of reducing inflammation in a subject in need thereof may include administering to the subject an effective amount of anacardic acid (AA) or a pharmaceutically acceptable salt thereof. In an exemplary aspect, the method may include administering an effective amount of anacardic acid (AA) of Structure (I) above. In some aspects, the method may include administering to the subject an effective amount of AA or a pharmaceutically acceptable salt thereof.

In some aspects, inflammatory conditions may include any disease, disorder, or condition including chronic inflammation related to persistent and dysregulated immune response. In some aspects, the inflammatory condition may be related to autoimmune disorders or immune-mediated disorders involving inappropriate immune responses. In an exemplary aspect, inflammatory conditions including autoimmune disorders or conditions may include, but not be limited to, rheumatoid arthritis, multiple sclerosis, optic neuritis. In an exemplary aspect, inflammatory conditions include immune-mediated disorders or conditions, which may include, but not be limited to, inflammatory bowel disease and psoriasis.

In some aspects, the inflammatory condition may include dysregulation of certain T lymphocytes, including TH1, TH17, T regulatory cells, or a combination thereof. In some aspects, the inflammatory condition may include dysregulation of cytokines, including TNF-α, IL-6, and IL-17 pro-inflammatory cytokines.

In some aspects, the method of treating an inflammatory condition may include administering anacardic acid (AA) or a pharmaceutically acceptable salt thereof, which may reduce the expression of IL-17 cytokines in lymphoid cells of the body. Certain of the inflammatory conditions may be mediated by IL-17 cytokine expression, including in autoimmune disorders.

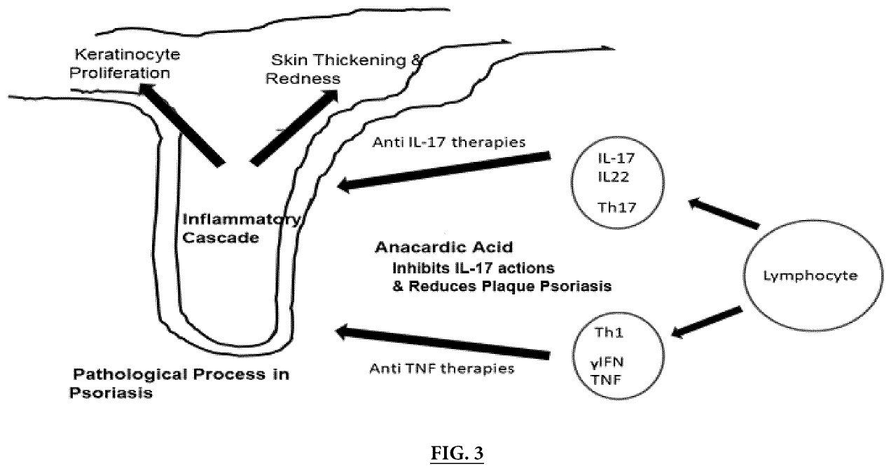

FIG. 3 depicts a pathway by which anacardic acid may inhibit expression of IL-17 and provide therapeutic effects on plaque psoriasis. This pathway provides that methods and compositions according to the present disclosure may provide biologic agent which target IL-17, TNF alpha cytokines, or their receptors and thus are useful in the treatment of psoriasis. By way of experimentation, methods of administration and compositions according to aspects of the present disclosure, including administration of a saturated form of anacardic acid (AA), include in vivo marked reduction of the development of clinical paralysis mice induced to develop TH17 autoimmunity to neural antigens. Accordingly, anacardic acid (AA) compositions and methods of treatment utilizing anacardic acid may inhibit the development of experimental autoimmune diseases mediated by TH17 cells.

Methods of treatment and compositions according to aspects of the present disclosure may reduce production of IL-17 in lymphocytes, with such reduction confirmed through in vitro experimentation of administration of a synthetic form of saturated anacardic acid. By way of experimentation, spleen derived lymphocytes were stimulated in vitro culture with antiCD3/antiCD28 antibodies for three days. At the end of three days, the cell culture supernatants were examined for the reduction of expression of IL-17 in the cell culture supernatants using enzyme linked assays (ELIZA) and by quantitative PCR (qPCR) from the mRNA extracted from the cell pellets. The methods of treatment and administration of the compositions of the present disclosure resulted in a 43% inhibition of IL-17 in cell culture supernatants following addition of 20 μM of saturated anacardic acid using ELIZA and 43.5% inhibition of IL-17 by using qPCR methods.

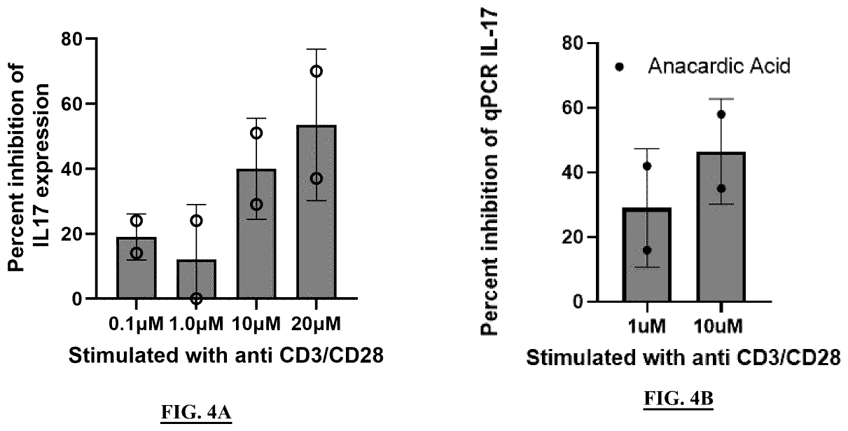

FIGS. 4A-4B depict quantification of IL-17 inhibition according to methods and compositions of the present disclosure. As shown by FIG. 4A, the expression of IL-17 by ELIZA was inhibited in the presence of anacardic acid, with the Y-axis providing a relative percent decrease in IL-17 levels when compared to controls. As shown by FIG. 4B, the mRNA expression of IL-17 in spleen cells was inhibited when such cells were cultured in the presence of anacardic acid.

In some aspects, the method may include administering a composition intramuscularly, subcutaneously, intravenously, intrathecally, intranasally, intradermally, orally, or some combination thereof.

In some aspects, the composition may be administered according to a treatment plan. In an exemplary aspect, the composition may be administered daily, every other day, weekly, monthly, or any interval of time falling therebetween.

In some aspects, a composition to be used to treat an inflammatory condition in a subject may include an anacardic acid (AA) or a pharmaceutically acceptable salt thereof as an active agent. In some aspects, the composition may further include a vegetable oil as a pharmaceutically acceptable carrier or as a diluent. In an exemplary aspect, the pharmaceutically acceptable carrier or the diluent may include corn oil, coconut oil, peanut oil, mustard oil, sesame oil, and/or combinations thereof. The vegetable oil, including all disclosed oils, may further include a refined vegetable oil for higher purity, consistency, stability, safety, better absorption properties, and lower risk of interaction with the active agent.

In some aspects, the composition may include the anacardic acid (AA) or a pharmaceutically acceptable salt thereof in a range of 0.001%-1.00% as the active agent. The composition may further include the pharmaceutically acceptable carrier or the diluent as a remainder of the composition in addition to the active agent. Unless otherwise indicated herein, the concentration of the active agent in the composition may be expressed as a w/v % (weight/volume percent). In certain aspects, the composition may include AA or a pharmaceutically acceptable salt thereof in a range of 0.01%-1.00%, 0.10%-1.00%, 0.005%-1.00%, 0.05%-1.00%, 0.5%-1.00%, 0.001%-0.9.%, 0.001%-0.8%, 0.001%-0.7%, 0.001%-0.6%, 0.001%-0.5%, 0.001%-0.4%, 0.001%-0.3%, 0.001%-0.2%, 0.001%-0.1%, 0.001%-0.09%, 0.001%-0.08%, 0.001%-0.07%, 0.001%-0.06%, 0.001%-0.05%, 0.001%-0.04%, 0.001%-0.03%, 0.001%-0.02%, 0.001%-0.01%. In certain aspects, the composition may include AA or a pharmaceutically acceptable salt thereof having a w/v % of 0.001%, 0.002%, 0.003%, 0.004%, 0.005%, 0.006%, 0.007%, 0.008%, 0.009%, 0.01%, 0.02%, 0.03, 0.04%, 0.05%, 0.06%, 0.07%, 0.08%, 0.09%, 0.015%, 0.025%, 0.035%, 0.045%, 0.055%, 0.065%, 0.075%, 0.085%, 0.095%, 0.1%, 0.2%, 0.3%, 0.4%, 0.5%, 0.6%, 0.7%, 0.8%, 0.9%, 0.15%, 0.25%, 0.35%, 0.45%, 0.55%, 0.65%, 0.75%, 0.85%, 0.95% 0.96%, 0.97%, 0.98%, 0.99%, 1.00%.

In some aspects, the composition may be prepared as a stock solution to be diluted as needed for formulation and administration to a subject in need thereof. In an exemplary aspect, the composition may be provided as a 1% stock solution.

In some aspects, the composition may include the anacardic acid (AA) or a pharmaceutically acceptable salt thereof in a range of about 1-2 grams dissolved in about 100 cc of the pharmaceutically acceptable carrier or diluent as the stock solution. In an exemplary aspect, the composition may include 1-2 grams of the anacardic acid (AA) or a pharmaceutically acceptable salt thereof dissolved in 100 cc of the vegetable oil as a stock solution. Further, the stock solution may be diluted to a range of a 1:10-1:20 dilution. In an exemplary aspect, the 1:10-1:20 dilution may provide the composition in a therapeutic strength appropriate for administration to a patient in need thereof. The 1:10 dilution may include a concentration of the active agent of 1-2 mg/mL. the 1:20 dilution may include a concentration of the active agent of 0.5-1.0 mg/mL. Dilutions of a stock solution to form the composition may include diluting the stock solution with the same pharmaceutically acceptable carrier or diluent as used to prepare the stock solution. In some aspects, a dilution formed from the stock solution may include a pharmaceutically acceptable carrier or diluent different from the pharmaceutically acceptable carrier or diluent used to provide the stock solution.

The AA compounds for use in the methods described herein may be synthesized by routes known in the art and processes analogous to those well known in the chemical arts, particularly in light of the description contained herein. Satoh, et al., Chem Pharm Bull (Tokyo), 49 (1):18-22 (2001); Swamy et al., Chem. Commun. (Camb.), 54 (90):12758-61 (2018). AA and other starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.) and similar texts know to those skilled in the art.

The purification of anacardic acid, including cardols and cardonols, has been performed from cashew nut shell oil. The anacardic acid was isolated as the calcium salt and the acid free cashew nut shell liquid was treated with ammonia and extracted with hexane and ethyl acetate mixture. In addition, anacardic acid has been isolated from cashew nut shell oil using supercritical carbon dioxide which avoids the use of organic solvents. Balasubramanyam et al., J Biol Chem, 278 (21):19134-40 (2003); Hemshekhar et al., Basic Clin Pharmacol Toxicol, 110 (2):122-32 (2012); Philip et al., J Agric Food Chem, 56 (20):9350-54 (2008).

In some aspects, the composition may be provided in the form of a gel, ointment, lotion, paste, cream, mousse, liquid, spray, suspension, or dispersion. In exemplary aspects, the composition may be provided in the form of a dosage unit containing a particular amount of the composition.

In some aspects, the composition may be used for treatment of inflammatory bowel disease, rheumatoid arthritis, multiple sclerosis, optic neuritis, and inflammatory disorders of skins. In some aspects, inflammatory disorders of the skin may include psoriasis, dermatitis, UV induced skin burn, lupus, vitiligo, scleroderma, dermatomyositis, epidermolysis bullosa, bullous pemphigoid, leukoderma, Koebner's phenomenon, and any other skin disorder.

FIG. 2 depicts an exemplary Scheme 1 for a method of preparing an anacardic acid (AA) according to an aspect of the present disclosure. In some aspects, a method of preparing the anacardic acid (AA) or a pharmaceutically acceptable salt thereof may include preparation of a highly pure anacardic acid (AA).

The method may include a step (i) of reacting by a diazotization reaction of a 2-amino 6-methyl benzoic acid to obtain a 2-hydroxy-6-methyl-benzoic acid.

The method may further include a step (ii) of protecting the hydroxy and acid group of the 2-hydroxy-6-methyl-benzoic acid of step (i) by reacting with halomethyl methyl ether in the presence of a base to obtain a 2-methoxymethoxy-6-methyl-benzoic acid methoxymethyl ester. In an exemplary aspect, the step (ii) of the method may include providing the halomethyl methyl ether is selected from chloromethyl methyl ether, fluoromethyl methyl ether or iodomethyl methyl ether. In an exemplary aspect, the step (ii) of the method may include providing the base is selected from NaOH, KOH, or NH₄OH.

The method may further include a step (iii) of reacting the 2-methoxymethoxy-6-methyl-benzoic acid methoxymethyl ester of step (ii) with a 1-iodo tetradecane in the presence of Lithium diisopropylamine (LDA) to obtain the anacardic acid.

In some aspects, the method provided in Scheme 1 may produce a saturated form of anacardic acid (AA). In an exemplary aspect, the method may further include a step of purifying the saturated anacardic acid (AA) by a recrystallization method. The recrystallization method to purify the saturated anacardic acid (AA) may include using an acetonitrile solvent.

As used herein, the phrase “one or more of,” when used with a list of items, means that different combinations of one or more of the items may be used and only one of each item in the list may be needed. For example, “one or more of” item A, item B, and item C may include, for example, without limitation, item A or item A and item B. This example also may include item A, item B, and item C, or item Band item C.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

As used herein, substantially or highly pure means sufficiently homogeneous to appear free of readily detectable impurities, as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. A substantially chemically pure compound can, however, be a mixture of stereoisomers or isomers. In such instances, further purification might increase the specific activity of the compound.

Thus, it is seen that the apparatus and methods of the present disclosure readily achieve the ends and advantages mentioned as well as those inherent therein. While certain preferred embodiments of the disclosure have been illustrated and described for present purposes, numerous changes in the arrangement and construction of parts and steps may be made by those skilled in the art, which changes are encompassed within the scope and spirit of the present disclosure as defined by the appended claims. Each disclosed feature or embodiment may be combined with any of the other disclosed features or embodiments.

What is claimed is:

1. A method of reducing inflammation in a subject in need thereof, wherein the method comprises administering to the subject an effective amount of anacardic acid (AA) having a structure: 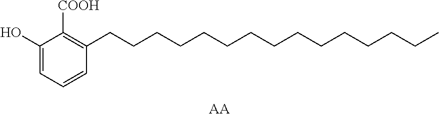 [SMILES: CCCCCCCCCCCCCCCc1cccc(O)c1C(=O)O ; "AA"]
or a pharmaceutically acceptable salt thereof.

2. The method of claim 1, wherein AA or a pharmaceutically acceptable salt thereof is administered to the subject.

3. The method of claim 1, wherein the subject has been diagnosed as having an inflammatory disorder.

4. The method of claim 3, wherein the inflammatory disorder is inflammatory bowel disease, rheumatoid arthritis, multiple sclerosis, optic neuritis, inflammatory disorders of the skin, or combinations thereof.

5. The method of claim 3, wherein the inflammatory disorder is psoriasis.

6. The method of claim 1, wherein AA or a pharmaceutically acceptable salt thereof is administered with a pharmaceutically acceptable carrier.

7. The method of claim 6, wherein AA is administered topically, and the pharmaceutically acceptable carrier is a vegetable oil.

8. The method of claim 7, further comprising administering a composition comprising the effective amount of AA or a pharmaceutically acceptable salt thereof wherein the composition comprises 0.001%-1.00% of AA.

9. The method of claim 7, wherein the vegetable oil is a corn oil, coconut oil, peanut oil, mustard oil, sesame oil, or combinations thereof.

10. The method of claim 7, further comprising administering a composition comprising the effective amount of AA or a pharmaceutically acceptable salt thereof of 0.5-2 mg/mL.

11. The method of claim 10, wherein administering a composition comprising the effective amount of AA or a pharmaceutically acceptable salt thereof of 0.5-1 mg/mL.

12. The method of claim 10, wherein administering a composition comprising the effective amount of AA or a pharmaceutically acceptable salt thereof of 1-2 mg/mL.

13. A method of reducing inflammation in a subject in need thereof, wherein the method comprises administering to the subject an effective amount of anacardic acid (AA) or a pharmaceutically acceptable salt thereof.

14. The method of claim 13, wherein the subject has been diagnosed as having an inflammatory disorder.

15. The method of claim 14, wherein the inflammatory disorder is psoriasis.

16. The method of claim 14, wherein the inflammatory disorder is inflammatory bowel disease, rheumatoid arthritis, multiple sclerosis, optic neuritis, inflammatory disorders of the skin, or combinations thereof.

17. The method of claim 13, wherein AA or a pharmaceutically acceptable salt thereof is administered with a diluent.

18. The method of claim 17, wherein AA is administered topically, and the diluent is a vegetable oil.

19. The method of claim 13, further comprising administering a composition comprising the effective amount of AA or a pharmaceutically acceptable salt thereof wherein the composition comprises 0.001%-1.00% of AA.

20. The method of claim 13, further comprising administering a composition comprising the effective amount of AA or a pharmaceutically acceptable salt thereof of 0.5-2 mg/mL.
